(19) United States
(12) Patent Application Publication
(10) Pub. No.: US 2022/0390472 A1
(43) Pub. Date: Dec. 8, 2022

(54) Synchronous Detection Method of Medicaments Influencing ARR in the Detection Process of Renin Activity by Liquid Chromatography-Tandem Mass Spectrometry

(71) Applicant: Hangzhou Calibra Diagnostics Co., Ltd., Hangzhou (CN)

(72) Inventors: Pengyun LIU, Hangzhou (CN); Ziqing KONG, Hangzhou (CN); Jinfei MA, Hangzhou (CN); Xiaofen YUAN, Hangzhou (CN); Huafen LIU, Hangzhou (CN)

(21) Appl. No.: 17/494,444

(22) Filed: Oct. 5, 2021

(30) Foreign Application Priority Data
Jun. 2, 2021 (CN) 2021106145678

(51) Int. Cl. G01N 33/74; G01N 30/72

(52) U.S. Cl. CPC G01N 33/743; G01N 30/7233; G01N 2800/321

(57) ABSTRACT

The present invention provides a synchronous detection method of medicaments influencing ARR in the detecting process of renin activity by liquid chromatography-tandem mass spectrometry. The method achieves the qualitative screening of medicaments influencing ARR values while detecting plasma renin activity by liquid chromatography-tandem mass spectrometry; moreover, the method can be used to assist to analyze and judge ARR as negative, positive, false negative or false positive. The detecting method achieves effective extraction of angiotensin I and 43 hypertension therapeutics synchronously through protein precipitation to samples, and enables to perform synchronous detection of a plurality of indices including angiotensin I and 43 hypertension therapeutics on the extracted sample by liquid chromatography-tandem mass spectrometry technology of high throughput, high specificity and high sensitivity. Moreover, the detection method utilizes the MRM mass spectrum parameters for preliminary screening of the medicaments, and rechecks the medicaments according to the retention time thereof. The ARR detection value is combined with the medicament screening result for co-analysis, which effectively distinguishes the false positive or false negative result of the detection while avoiding the patient's withdrawal of medicament for treatment, thereby improving the detection accuracy of the actual primary aldosteronism, and facilitating clinical promotion and application.

### TECHNICAL FIELD

The present invention belongs to the technical field of biochemical analysis; and particularly relates to a qualitative detection method of primary aldosteronism, and especially relates to a method for detecting and judging a ratio of plasma aldosterone concentration (PAC)/plasma renin activity (PRA), which is also called ARR.

### BACKGROUND

Primary aldosteronism (PA, abbreviated for primary aldosteronism) is a common endocrine hypertension, and is characterized in independent secretes of aldosterone by adrenal cortex, resulting in sodium retention and potassium removal in the body, increase of blood volume, and inhibition in activity of renin-angiotensin system; and it is mainly manifested as hypertension and hypokalemia clinically.

The ratio of plasma aldosterone concentration (PAC)/plasma renin activity (PRA), which is ARR, is the most reliable method for the screening of primary aldosteronism. PRA is an index to measure the production efficiency of angiotensin I in peripheral blood under the catalysis of renin, and has great significance in the typing diagnosis of RAAS-caused hypertension.

In 2010, directed by the Endocrinology Branch of the Chinese Medical Association conducted PA screening to 1656 cases of refractory hypertension patients in 19 centers of 11 provinces across the country; and it was reported that the morbidity was 7.1%. Researches published by professor Li Qifu's team have indicated that the incidence of PA in newly diagnosed hypertension exceeds 4.0%. In the 2020 version of Expert Consensus on Diagnosis and Treatment of Primary Aldosteronism, ARR serves as the optimal screening index of PA, and it believes that it is of practical guiding significance for clinical work to carry out PA screening to hypertension, particularly refractory hypertension and the people newly diagnosed with hypertension, and all the patients newly diagnosed with hypertension are recommended to receive PA screening.

It is further pointed out in the Expert Consensus that ARR values will be influenced by multiple factors including age, sex, diet, medicaments to be used, position, serum potassium and creatinine, probably resulting in a false positive or false negative result, therefore the therapeutics use must be concerned. It is required in the Consensus that ARR detection must be conducted for at least four weeks after withdraw of the medicament influencing ARR greatly, thus avoiding a false positive or false negative result; what calls for special attention is that ACE inhibitors (ACE-Is), dihydropyridine calcium channel blockers (CCBs) and angiotensin II receptor blockers (ARBs) can decrease PAC and sharply increase PRA level, thus reducing ARR in particular, resulting in a false negative detection result; therefore, the detection is performed after withdraw the above medicament for at least 2 weeks. But PA often causes severe hypertension; therefore, withdrawal of the medicament has potential hazard to the patient.

ARR determination includes the detection of plasma aldosterone concentration (PAC) and plasma renin activity (PRA); currently, PRA is generally determined by radioimmunoassay to indirectly reflect the level of plasma renin concentration by a rate that angiotensinogen is transformed into angiotensin I within unit time and unit volume. Majority of centers take radioimmunoassay to determine PAC. But there are inherent defects of immunoassay, such as, poor specificity and the presence of cross coupling reaction to the determination of renin activity and aldosterone content by immunoassay; and immunoassay cannot achieve the screening of hypertension therapeutics influencing ARR values, and moreover, patients need to face the risk of medicament withdrawal for detection.

Therefore, it is in urgent need of finding out a simpler and more convenient ARR detection method, which can not only avoid patient's withdrawal of the medicament, but also can effectively reduce the false positive or false negative result of the detected PA, thereby improving the detecting accuracy of ARR and renin activity in clinical practice.

### SUMMARY OF THE INVENTION

Directed to the problem existing in the prior part, the present invention provides a synchronous screening method of medicaments influencing ARR in the detection process of renin activity by liquid chromatography-tandem mass spectrometry. The method achieves the qualitative screening of medicaments influencing ARR values while detecting plasma renin activity by liquid chromatography-tandem mass spectrometry. The ARR detection value is combined with the screening results of interfering medicaments for co-analysis, which effectively reduces the false positive or false negative result of the detection while avoiding patient's withdrawal of the medicament, thereby improving the detection accuracy of the actual primary aldosteronism, and facilitating clinical promotion and application.

For the first aspect, the present invention provides a method for synchronous qualitative detection of medicaments influencing ARR in the detection process of renin activity by liquid chromatography-tandem mass spectrometry. The method can achieve the qualitative screening of the hypertension therapeutics influencing ARR in plasma while detecting the content of angiotensin I in plasma samples; and the method can be used to synthetically judge and analyze ARR as one of a negative, positive, false negative or false positive result in combination with the influences of positive screening medicaments on ARR and a concentration of aldosterone.

In some embodiments, there are 43 commonly used hypertension therapeutics influencing ARR, including 9 β-receptor blockers, 5 potassium-removing diuretics, 3 potassium-retaining diuretics, 10 angiotensin converting enzyme inhibitors, 6 angiotensin receptor antagonists, 8 calcium ion antagonists, 1 central α2 receptor agonist and 1 non-steroid anti-inflammatory drug.

Based on the 2020 version of Expert Consensus on Diagnosis and Treatment of Primary Aldosteronism, the reasons for the medicament causing ARR false positive or false negative are shown in the table below:

| | | Influence | |
|---|---|---|---|
| | Influence on | on renin | Influence |
| Factor | aldosterone | activity | on ARR |
| Medicament factors | | | |
| β-receptor blocker | ↓ | ↓↓ | ↑(False positive) |
| Centralα2receptor | ↓ | ↓↓ | ↑(False positive) |
| agonist | | | |
| NSAID | ↓ | ↓↓ | ↑(False positive) |
| Potassium-removing | →↑ | ↑↑ | ↓(False negative) |
| diuretics | | | |
| Potassium-retaining | ↑ | ↑↑ | ↓(False negative) |
| diuretics | | | |
| ACEI | ↓ | ↑↑ | ↓(False negative) |
| ARB | ↓ | ↑↑ | ↓(False negative) |
| Dihydropyridine CCB | →↓ | ↑ | ↓(False negative) |
| Serum potassium state | | | |
| Hypokalemia | ↓ | →↑ | ↓(False negative) |
| Hyperkalemia | ↑ | →↓ | ↑(False positive) |
| Sodium salt intake | | | |
| Low sodium diet | ↑ | ↑↑ | ↓(False negative) |
| High sodium diet | ↓ | ↓↓ | ↑(False positive) |
| Growth of age | ↓ | ↓↓ | ↑(False positive) |
| Other factors | | | |
| Renal insufficiency | → | ↓ | ↑(False positive) |
| Pregnancy | ↑ | ↑↑ | ↓(False negative) |
| Renal vascular | ↑ | ↑↑ | ↓(False negative) |
| hypertension | | | |
| Malignant hyper tension | ↑ | ↑↑ | ↓(False negative) |

Note:
ARR: a ratio of plasma aldosterone torenin activity; ACEI: angiotensin converting enzyme inhibitor; ARB: an4giotensin receptor inhibitor; and CCB: calcium channel blockers.

Moreover, it is stipulated in the 2020 version of Expert Consensus on Diagnosis and Treatment of Primary Aldosteronism, ARR needs to be measured for at least four weeks after withdrawing the medicament greatly influencing ARR values, thus avoiding a false positive or false negative result; and for ACEI, ARB, CCB, and other medicaments, the detection needs to be conducted for two weeks after the withdrawal of the medicament, thus avoiding a false negative result. But PA often causes severe hypertension; therefore, withdrawal of the medicament has potential hazard to the patient.

Through marketing survey, the research team of the present invention has collected 43 commonly-used hypertension therapeutics with huge influences on ARR and a higher frequency of usage, and established a liquid chromatography-tandem mass spectrometry method, which inventively achieves the synchronous detection of the medicaments influencing ARR during the detection of renin activity. Therefore, the present invention avoids the risk of medicament withdrawal or stop, improves the judgment accuracy of ARR in clinical practice, and effectively reduces the false positive or false negative result of the detected PA, and thus has great significance of clinical application.

Further, the ARR has a computing formula as follows: ARR=concentration of aldosterone/production rate of angiotensin I.

PAC during the detection of ARR in the present invention is detected by liquid chromatography-tandem mass spectrometry after performing pretreatment of a plasma sample with solid-liquid extraction (SLE).

Further, the hypertension therapeutics influencing ARR are respectively:

the 9 β-receptor blockers include Arotinolol, Atenolol, Bisoprolol, Esmolol, Labetalol, Metoprolol, Nebivolol, Propranolol, and Sotalol;

the 5 potassium-removing diuretics include Bumetanide, Furosemide, Hydrochlorothiazide, Indapamide, and Torasemidel;

the 3 potassium-retaining diuretics include Amiloride, Eplerenone, and Spironolactone;

the 10 angiotensin converting enzyme inhibitors include Benazepril, Captopril, Enalapril, Fosinopril, Imidapril, Lisinopril, Perindopril, Quinapril, Ramipril, and Trandolapril;

the 6 angiotensin receptor antagonists include Candesartan, Irbesartan, Losartan, Olmesartan, Telmisartan, and Valsartan;

the 8 calcium channel blockers include Amlodipine, Benidipine, Felodipine, Flunarizine, Lercanidipine, Nicardipine, Nifedipine, and Nimodipine;

the 1 central α2 receptor agonist includes Methyldopa; and

Further, the β-receptor blocker, central α2 receptor agonist, and non-steroid anti-inflammatory drug can increase ARR to show a false positive result; the potassium-removing diuretics, potassium-retaining diuretics, angiotensin converting enzyme inhibitors, angiotensin receptor antagonists, and calcium ion antagonists can decrease ARR to show a false negative result.

Any one of β-receptor blocker, central α2 receptor agonist, and non-steroid anti-inflammatory drug can decrease the aldosterone concentration and renin activity of a patient; where the decrease extent of renin activity is greater than that of aldosterone concentration, thereby leading to a false positive result.

Potassium-removing diuretics or potassium-retaining diuretics can increase the aldosterone concentration and renin activity of a patient; where the increase extent of renin activity is greater than that of aldosterone concentration; any one of the angiotensin converting enzyme inhibitor, angiotensin receptor antagonist and calcium ion antagonist can decrease aldosterone concentration, but increase renin activity, thereby leading to a false negative result.

Further, the analysis steps of ARR values are as follows: a clinical medication AI guidance system is used to analyze and judge ARR as one of a negative, positive, false negative or false positive result; and the clinical medication AI guidance system comprises a patient information module, a database module and a decision tree system.

Further, the information module is used to record a patient's basic information; the database module is used to store detection results of ARR and detection results of hypertension therapeutics; and the decision tree system automatically analyzes and judges an ARR index as one of a negative, positive, false negative or false positive result according to the detection results.

Further, the judgment cut-off value of ARR is 30 when the PA is analyzed and judged as one of a negative, positive, false negative, or false positive result according to the detection results.

Further, the analysis and judgment method of the decision tree system is as follows: when the ARR detection result is slightly lower than a negative result of 30, and meanwhile if the patient is detected to contain a medicament capable of decreasing ARR, the patient may be judged as false negative, and thereby needs to receive reexamination after withdrawal of the medicament or further receive an experiment for PA definite diagnosis; when the ARR detection result is lower than a negative result of 30, and meanwhile if the patient is detected to contain a medicament capable of increasing ARR, the patient may be judged as negative; and when the ARR detection result is a positive result greater than 30, and meanwhile if the patient is detected to contain a medicament capable of increasing ARR, the patient may be judged as false positive, and thereby needs to receive reexamination after withdrawal of the medicament or further receive an experiment for PA definite diagnosis; and when the ARR detection result is a positive result greater than 30, and meanwhile if the patient is detected to contain a medicament capable of decreasing ARR, the patient may be judged as positive.

Further, the production rate of the angiotensin I is obtained by detecting concentrations of angiotensin I in the sample before and after incubation and according to the following formula: production rate of angiotensin I=(concentration of angiotensin I after incubation−concentration of angiotensin I before incubation)/incubation time.

Further, the qualitative detection method of PA has the following specific detection steps:

(1) sample pretreatment: 1) taking two parts of parallel 100 μL samples to be detected, and respectively adding a generating buffer, mixing evenly, subjecting one part to incubation, and subjecting another part to the next operation directly without incubation; 2) adding a terminating solution to the samples where the generation buffer solution is added for mixing evenly; 3) adding a protein precipitant containing an internal standard following addition of terminating solution and vortex mixing evenly; and 4) performing centrifugation, and taking supernatant for detection on an instrument;

(2) Quantitating analysis of angiotensin I to the solution obtained in the above step with a high performance liquid chromatography-tandem mass spectrometry system; and synchronously screening the 42 hypertension therapeutics influencing ARR with non-incubated samples;

(3) check the screening result of the medicament; when there's a positive medicament result, retention time of the medicament must be compared to the retention time of the positive hypertension therapeutic in a “mixed medicament-screening working solution”, thus ensuring the accuracy of the positive result of the medicament screening; with respect to the sample with positive ARR result, a check is needed whether there's any positive screening result with therapeutic medicament which could result in increase of ARR; with respect to the sample with negative ARR result, a check is needed whether there's any positive screening result with therapeutic medicament which could result in decrease of ARR;

Further, the protein precipitant containing internal standard is prepared by: methanol, a zinc sulfate aqueous solution and an angiotensin I isotope-labelled internal standard.

Further, the generation buffer is: a PMSF-containing Tris and EDTA buffer solution;

Further, the terminating solution is: formic acid or acetic acid.

Further, the mixed medicament-screening working solution is a solution prepared by standard substances of the 43 commonly-used hypertension therapeutics influencing ARR and used to detect a lower limit concentration.

Further, preparation of standard curve: a standard substance of angiotensin I is prepared to calibrators in BSA aqueous solution at different concentrations.

Currently, ARR is generally detected by radioimmunoassay. Radioimmunoassay cannot achieve the screening of multiple therapeutics through a single detection, and if a patient withdraws the medicament in accordance with the provision before ARR detection, it is difficult to assess the influences of antihypertensive medicaments on ARR detection.

Through studies, the present invention proves that the method can achieve synchronous detection of the 43 hypertension therapeutics influencing ARR in the detection of PRA by liquid chromatography-tandem mass spectrometry; that is, the method achieves more accurate judgment to ARR via a one-step method without the withdrawal of medicament. Moreover, a large number of clinical experiments prove that the method has accurate and reliable detection results, and is highly desirable in large-scale clinical popularization and application.

During the quantitative detection of renin activity and synchronous detection of hypertension therapeutics influencing ARR, the present invention utilizes a protein precipitation method for sample pretreatment to synchronously achieve the effective extraction of angiotensin I and hypertension therapeutics. Moreover, the extracted sample is subjected to the synchronous detection of a plurality of indices by the high throughput characteristic of liquid chromatography-tandem mass spectrometry, which effectively screens the 43 therapeutics influencing ARR while detecting the concentration of angiotensin I. The ARR detection value is combined with the screening result of the interfering medicaments for co-analysis, which effectively reduces the false rate of the detection while avoiding patient's medicament withdrawal, thereby improving the judgement accuracy of ARR and lowering the patient's risk.

The detection method provided by the present invention requires a small amount of samples; and 100 μL plasma sample can detect the content of angiotensin I in plasma before incubation, and can achieve the qualitative screening of the multiple antihypertensive medicaments influencing ARR values.

The sample pretreatment provided by the present invention is protein precipitation, without complicated liquid-liquid extraction or SPE process. Due to huge difference in physical characteristic, complicated pretreatment could easily result in poor extracting efficiency to part of the medicaments while protein precipitation could avoid the decline of extraction rate. Therefore, the invention can achieve effectively high throughput treatment and detection of the sample.

Further, for mass spectrometric detection, a triple quadrupole mass spectrometer with a model of SCIEX 4500MD is used; and the mass spectrometric detection is performed by adopting a positive ion mode of an electrospray ionization ion source (ESI+) and a multiple-reaction monitoring (MRM) mode.

In the detection method provided by the present invention, the running time for each sample is only 3.8 min.

Further, the incubation refers that a sample mixed with generation buffer is put to a 37° C. water bath for 3 h; and a non-incubated sample is used for screening of the 43 hypertension therapeutics influencing ARR; and a mobile phase A of the liquid chromatography is an aqueous solution with 0.1% FA, mobile phase B is a methanol solution with 0.1% formic acid, and gradient elution is performed.

Further, influences of the 43 hypertension medicaments on ARR can be analyzed and predicted by AI software.

On the other hand, the present invention provides a kit for synchronous qualitative detection of antihypertensive medicaments influencing ARR in the detecting process of renin activity by liquid chromatography-tandem mass spectrometry; the kit includes a protein precipitant with internal standard, a generating buffer solution, a terminating solution, a mixed medicament-screening working solution and mobile phases additives; wherein the protein precipitant with internal standard is prepared by: methanol, a zinc sulfate aqueous solution and an angiotensin I isotope-labelled internal standard; the generating buffer solution is: a PMSF-containing Tris and EDTA buffer solution; and the terminating solution is: formic acid or acetic acid; the mixed medicament-screening working solution is: a mixed medicament-screening working solution prepared by standard substances of the 43 hypertension therapeutics influencing ARR, which is mixed at certain concentration; mobile phase A of the liquid chromatography is a aqueous solution with 0.1% formic acid, and mobile phase B is a methanol solution with 0.1% formic acid.

In a further aspect, the present invention provides a detection method of renin activity, and the method is mainly implemented by taking the above quantitative detection of angiotensin I and qualititative screening of hypertension therapeutics influencing ARR in a plasma sample, analyzing the actual content of renin activity according to the detection results.

In a further aspect, the present invention provides a method for synchronous detection of 43 hypertension medicaments influencing ARR in the detecting process of renin activity in a plasma sample by liquid chromatography-tandem mass spectrometry in clinical judgment of ARR values and renin activity.

Any one of β-receptor blockers, central α2 receptor agonists, and non-steroid anti-inflammatory drugs can decrease patients' renin activity; the potassium-removing diuretics, and potassium-retaining diuretics can increase patients' renin activity; any one of angiotensin converting enzyme inhibitors, angiotensin receptor antagonists and calcium channel blockers can increase patients' renin activity and result in decrease of ARR. Therefore, the synchronous detection method of the 43 hypertension medicaments influencing ARR in the detection process of renin activity in a plasma sample by liquid chromatography-tandem mass spectrometry simultaneously can provide more accurate judgment for the ARR curoff.

The present invention has the following beneficial effects:

(1) The present invention achieves the qualitative screening of the 43 hypertension therapeutics influencing ARR values while detecting PRA; ARR is combined with the screening results of the interfering medicaments for co-analysis, which effectively reduces the false positive or false negative result of the ARR detection while avoiding patients' withdrawal of medication therapy, thereby improving the detection accuracy of renin activity in clinical practice, and facilitating the clinical promotion and application.

(2) During the detection of renin activity, a protein precipitation method is used for sample pretreatment, which synchronously achieves the effective extraction of angiotensin I and the 43 hypertension therapeutics, thereby achieving the synchronous detection.

(3) The extracted sample is subjected to the synchronous detection of a plurality of indices by utilizing the characteristics of high throughput, high specificity and high sensitivity of the liquid chromatography-tandem mass spectrometry, which effectively screens the 43 therapeutics influencing ARR while detecting the content of angiotensin I.

(4) A small amount of sample is required; 100 μL plasma sample can be used to detect the content of angiotensin I in plasma before incubation, and can achieve the qualitative screening of the 43 antihypertensive medicaments influencing ARR.

(5) During the detection, MRM parameters of the screened medicaments are utilized for the preliminary screening of the therapeutics while performing quantitative determination of angiotensin I; and for the medicament whose preliminary screening result is positive, a doublecheck of a positive hit reference to the retention time of each therapeutic in a “mixed medicament-screening working solution”, thus ensuring the accuracy of the screening result of the medicament.

(6) The liquid chromatography-tandem mass spectrometry running time for each sample is only 3.8 min

(7) The present invention can provide an efficient and accurate screening method for PA therapeutic, and provide a practical and reliable basis for clinical diagnosis.

### BRIEF DESCRIPTION OF THE FICIURES

FIG. 1 is a detection flow of a sample in Example 1;

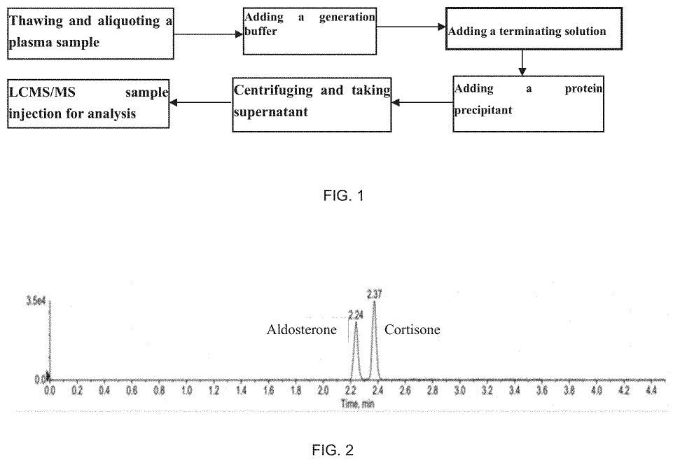

FIG. 2 shows a detection chromatogram of aldosterone in Example 1;

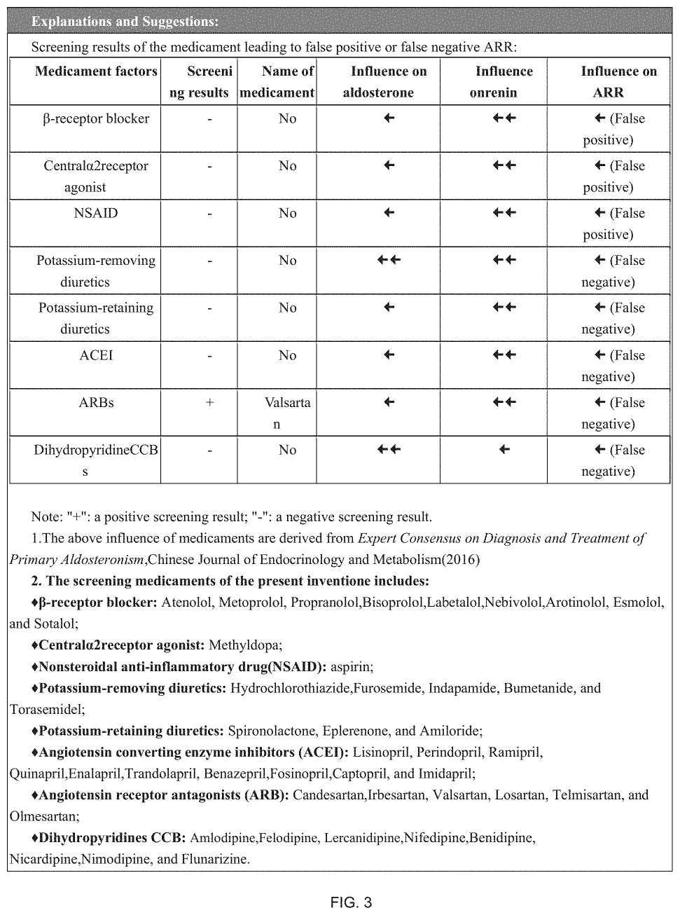

FIG. 3 is a screening report of primary aldosteronism in Example 1.

### DETAILED DESCRIPTION OF THE EMBODIMENTS

The present invention will be further described in detail with reference to the drawings and examples. It should be indicated that the examples below aim at facilitating the understanding of the present invention, but not limiting the present invention. Reagents used in the examples are known products, and are commercially available.

### EXAMPLE 1: QUALITATIVE DETECTION METHOD OF ARR PROVIDED BY THE PRESENT INVENTION

### I. Solution Preparation

Preparation of a ZnSO₄ solution: 3.56 g ZnSO₄.7H₂O were weighed, and dissolved into 40 mL water to prepare 89 mg/mL ZnSO₄.7H₂O aqueous solution. The volume of the solution could be adjusted according to the ratio as required, and the solution was stored at room temperature.

Generation buffer solution: 12.11 g trismetyl aminomethane (TRIS) and 7.4 g ethylenediamine tetraacetic acid (EDTA) were added to a 100 mL volumetric flask, and deionized water was added to 90 mL, the remaining solution was dissolved evenly under ultrasonic for 30 min. Deionized water was added to a scale line, and the solution was mixed evenly. Afterwards, the solution was transferred to a reservoir vessel made of polypropylene. pH was regulated within 5.45-5.50 by acetic acid, and the remaining solution was stored at −20° C. The preparation of generating buffer was performed on the same day of detection and analysis, 100 μL 100 mM PMSF solution (0.174 g PMSF was dissolved in 10 mL methanol) was added to a 10 mL generation buffer solution to get generation buffer solution (pH=5.4-5.6).

Terminating solution: formic acid or acetic acid.

A protein precipitant containing internal standard: 50 μL 1 μg/mL angiotensin I internal standard stock solution (methanol: water=1:1), and 100 μL 89 mg/mL zinc sulfate (ZnSO4.7H2O) were transferred to a 10 mL centrifugal tube, and 9850 μL methanol was added to the centrifugal tube, and mixed evenly to obtain a protein precipitant containing angiotensin I isotope-labeled internal standard.

A mixed medicament-screening working solution: standards of the 43 medicaments to be detected and partial metabolite standards were dissolved into DMSO, and prepared into a corresponding stock solution; and the stock solution was prepared into a mixed medicament-screening working solution having a LOD concentration based on the LOD concentration listed in the “Screening List and Limit of Detection of Medicaments Influencing ARR Values”.

### II. Sample Detection

the detection flow diagram was shown in FIG. 1.

(1) Reagent preparation: 20 μL generation buffer solution was added to two clean 1.2 mL 96-well collection plate first, and used for the following sample pretreatment.

(2) Sample thawing: a plasma sample to be detected was put to ice water (0° C.) and thawed out.

(3) Sampling: 2 parts of 100 μL parallel plasma samples were taken and transferred to two plates prepared in the step (1), then non-incubated and incubated samples were pretreated and detected respectively; and the rest samples were immediately frozen at −20° C.; and non-incubated sample was taken for the medicament screening.

(4) Incubated sample treatment: a batch of samples in step (3) were sealed by a silicone pad, and vortex mixed for a short time, then put to a 37° C. water bath for 3 h; and 12 μL terminating solution (formic acid) was added and vortex mixed, and 200 μL internal standard working solution containing 5 ng/mL angiotensin I isotope-labeled internal standard ANG I—¹³C¹⁵N was added and vortex mixed, afterwards, centrifuged for 10 min at 4° C. and 15000 rpm; 100 μL supernatant was transferred to a 96-well sample injecting plate for analysis by liquid chromatography-tandem mass spectrometry.

(5) Non-incubated sample treatment: another batch of samples remaining in step (3) were sealed by a sealing gasket and vortex mixed evenly. 12 μL terminating solution, formic acid was immediately added and vortex mixed, and 200 μL internal standard working solution containing an angiotensin I isotope internal standard was added, and vortex mixed evenly; afterwards, centrifuged for 10 min at 4° C. and 15000 rpm, 100 μL supernatant was transferred to a 96-well sample injection plate for analysis by liquid chromatography-tandem mass spectrometry (non-incubated samples were taken in the medicament screening, non-incubated samples could be not only used for the calculation of renin activity, but also for screening the medicament disturbing ARR).

(6) Calculation of renin activity: renin activity=production rate of angiotensin I, where the unit was ng/mL/hr, and calculating formula was: (concentration of angiotensin I after incubation−concentration of angiotensin I before incubation)/incubation time.

(7) During the liquid chromatography-tandem mass spectrometry, gradient elution was taken in liquid chromatography; and separation conditions of a substance to be detected was established by a reversed phase chromatography were as follows: the chromatographic column was Phenomenex C18 (2.6 μm, 50*2.1 mm), flow rate was 0.6 mL/min; column temperature was 40° C.; where mobile phase A was 0.1% formic acid aqueous solution, mobile phase B was a methanol solution of 0.1% formic acid; and volume ratio of mobile phase A to mobile phase B was 90-5%:10-95%. Gradient elution procedure was shown in Table 1; and the retention time of angiotensin I and its isotope-labeled internal standard was 1.92 min.

TABLE 1
Gradient elution procedure

| Time | Flow rate | Mobile phase | Mobile phase |
|---|---|---|---|
| (min) | (mL/min) | A (%) | B (%) |
| 0 | 0.6 | 90 | 10 |
| 1 | 0.6 | 90 | 10 |
| 2 | 0.6 | 5 | 95 |
| 3.2 | 0.6 | 5 | 95 |
| 3.25 | 0.6 | 90 | 10 |
| 3.8 | 0.6 | 90 | 10 |

The retention time of the standard substance of each hypertension therapeutic in the “mixed medicament-screening working solution” was shown in Table 2 below:

TABLE 2
Retention time of the standard substance
of each hypertension therapeutic

| | | Limit of | |
|---|---|---|---|
| | | detection | |
| | | (LOD) of | Retention- |
| Type of | Medicament | medicament | time |
| medicament | in English | (ng/mL) | (min) |
| Potassium- | Amiloride | 25 | 1.06 |
| retaining | | | |
| diuretics | | | |
| CCBs | Amlodipine | 25 | 2.19 |
| β-receptor | Arotinolol | 5 | 1.97 |
| blocker | | | |
| Non-steroid anti- | aspirin | 250 | 2.33 |
| inflammatory | | | |
| drug | | | |
| β-receptor | Atenolol | 25 | 0.65 |
| blocker | | | |
| ACEI | Benazepril | 5 | 2.19 |
| CCBs | Benidipine | 5 | 2.16 |
| β-receptor | Bisoprolol | 5 | 2.05 |
| blocker | | | |
| Potassium- | Bumetanide | 25 | 2.29 |
| removing | | | |
| diuretics | | | |
| ARBs | Candesartan | 25 | 2.32 |
| ACEI | Captopril | 100 | 1.95 |
| ACEI | Enalapril | 5 | 2.1 |
| Potassium- | Eplerenone | 25 | 2.16 |
| retaining | | | |
| diuretics | | | |
| β-receptor | Esmolol | 5 | 1.98 |
| blocker | | | |
| CCBs | Felodipine | 250 | 2.39 |
| CCBs | Flunarizine | 5 | 2.25 |
| ACEI | Fosinopril | 100 | 2.46 |
| Potassium- | Furosemide | 250 | 2.61 |
| removing | | | |
| diuretics | | | |
| Potassium- | Hydrochlorothiazide | 250 | 1.97 |
| removing | | | |
| diuretics | | | |
| ACEI | Imidapril | 5 | 2.13 |
| Potassium- | Indapamide | 25 | 2.17 |
| removing | | | |
| diuretics | | | |
| ARBs | Irbesartan | 5 | 2.27 |
| β-receptor | Labetolol | 25 | 2.04 |
| blocker | | | |
| CCBs | Lercanidipine | 5 | 2.26 |
| ACEI | Lisinopril | 25 | 1.77 |
| ARBs | Losartan | 5 | 2.26 |
| Centralα2 | Methyldopa | 250 | 0.39 |
| receptor agonist | | | |
| β-receptor | Metoprolol | 25 | 1.94 |
| blocker | | | |
| β-receptor | Nebivolol | 25 | 2.2 |
| blocker | | | |
| CCBs | Nicardipine | 5 | 2.15 |
| CCBs | Nifedipine | 250 | 2.27 |
| CCBs | Nimodipine | 25 | 2.34 |
| ARBs | Olmesartan | 25 | 2.12 |
| ACEI | Perindopril | 5 | 2.14 |
| β-receptor | Propranolol | 25 | 2.08 |
| blocker | | | |
| ACEI | Quinapril | 5 | 2.22 |
| ACEI | Ramipril | 5 | 2.2 |
| β-receptor | Sotalol | 25 | 0.53 |
| blocker | | | |
| Potassium- | Spironolactone | 250 | 2.29 |
| retaining | | | |
| diuretics | | | |
| ARBs | Telmisartan | 25 | 2.24 |
| Potassium- | Torasemide | 5 | 2.13 |
| removing | | | |
| diuretics | | | |
| ACEI | Trandolapril | 5 | 2.25 |
| ARBs | Valsartan | 100 | 2.34 |

During the mass spectrometric detection, a triple quadrupole mass spectrometer with a model of SCIEX 4500MD was used for the quantitative detection of angiotensin I and qualitative screening of antihypertensive medicaments before and after incubation; and the mass spectrometric detection was performed by adopting a positive ion mode of an electrospray ionization ion source (ESI+) and a multiple-reaction monitoring MRM mode; and the corresponding mass spectrometric detection method was configured as shown in Table 3 and Table 4:

TABLE 3
Parameter settings for mass spectrometric detection

| Mass Parameters | Value | |
|---|---|---|
| Curtain gas CUR | 30 | psi |
| Atomization gas GS1 | 50 | psi |
| Auxiliary heating gas | 50 | psi |
| GS2 | | |
| Heating temperature | 500° | C. |
| of ion sources | | |
| Collision gas CAD | 10 | psi |
| Spray voltage | 5500 | V |

TABLE 4
Mass spectrometry parameters of each substance to be detected

| | | | Dwell | | | |
|---|---|---|---|---|---|---|
| Compound | Q1 | Q3 | Time | DP | CE | CXP |
| ANG I-1 | 433.1 | 647.6 | 30 | 74 | 24 | 12 |
| ANG I-2 | 433.1 | 619.6 | 5 | 87 | 26 | 12 |
| ANG I-IS | 437.3 | 660.4 | 30 | 70 | 22 | 12 |
| Metoprolol | 268.1 | 133 | 4 | 90 | 37 | 9 |
| Esmolol | 296.1 | 145.1 | 4 | 78 | 38 | 9 |
| Labetolol | 329.2 | 162 | 4 | 78 | 35 | 9 |
| Bisoprolol | 326.5 | 116.2 | 4 | 96 | 25 | 9 |
| Flunarizine | 405.2 | 203 | 4 | 79 | 21 | 9 |
| Ramipril | 417.3 | 234.1 | 4 | 78 | 29 | 9 |
| Lercanidipine | 612.2 | 280.2 | 4 | 94 | 31 | 9 |
| Benazepril | 425.2 | 351.1 | 4 | 105 | 30 | 9 |
| Quinapril | 439.1 | 234 | 4 | 90 | 27 | 9 |
| Losartan | 423.2 | 207 | 4 | 69 | 33 | 9 |
| Trandolapril | 431.2 | 234 | 4 | 90 | 31 | 9 |
| Telmisartan | 515.4 | 276 | 4 | 153 | 59 | 9 |
| Nifedipine-1 | 347.1 | 315.1 | 4 | 79 | 13 | 9 |
| Nifedipine-2 | 347.3 | 195.1 | 4 | 65 | 58 | 6 |
| Nicardipine | 480.2 | 315.1 | 4 | 78 | 34 | 9 |
| Benidipine | 506.2 | 174 | 4 | 78 | 37 | 9 |
| Torasemide | 349.1 | 264.1 | 4 | 63 | 22 | 9 |
| Nebivolol | 406.1 | 151 | 4 | 105 | 41 | 9 |
| Indapamide | 366 | 132 | 4 | 78 | 20 | 9 |
| Imidapril | 406.2 | 234 | 4 | 75 | 27 | 9 |
| Eplerenone | 415.2 | 162.9 | 4 | 107 | 28 | 9 |
| Olmesartan | 447.1 | 207 | 4 | 83 | 37 | 9 |
| Irbesartan | 429.4 | 206.8 | 4 | 108 | 38 | 9 |
| Nimodipine | 419.2 | 343.1 | 4 | 82 | 13 | 9 |
| Valsartan-1 | 436.3 | 206.9 | 4 | 90 | 38 | 9 |
| Valsartan-2 | 436.4 | 207.2 | 4 | 90 | 38 | 4 |
| Felodipine | 384 | 338 | 4 | 76 | 13 | 9 |
| Spironolactone | 417.2 | 341.2 | 4 | 90 | 12 | 9 |
| Candesartan | 441.1 | 263 | 4 | 97 | 18 | 9 |
| Atenolol | 267.2 | 145 | 4 | 8 | 35 | 9 |
| Methyldopa | 212 | 166 | 4 | 75 | 21 | 9 |
| Amiloride | 230.1 | 170.9 | 4 | 69 | 24 | 9 |
| Sotalol | 273.1 | 133 | 4 | 70 | 36 | 9 |
| Arotinolol | 372 | 316.1 | 4 | 78 | 22 | 9 |
| Lisinopril | 406.2 | 84 | 4 | 78 | 52 | 9 |
| Enalapril | 377.2 | 234.1 | 4 | 70 | 26 | 9 |
| Perindopril | 369.1 | 172.1 | 4 | 90 | 31 | 9 |
| Propranolol | 260 | 116 | 4 | 90 | 24 | 9 |
| Amlodipine-1 | 409 | 237.9 | 4 | 69 | 12 | 9 |
| Amlodipine-2 | 409.2 | 206 | 4 | 55 | 37 | 10 |
| Fosinopril | 564.4 | 492.2 | 4 | 84 | 11 | 9 |
| Captopril-1 | 218.1 | 116.1 | 4 | 73 | 20 | 9 |
| Captopril-2 | 218.2 | 70 | 4 | 70 | 25 | 6 |
| Nifedipine Meta-1 | 331.2 | 270.2 | 4 | 165 | 47 | 9 |
| Felodipine Meta | 384 | 320 | 4 | 165 | 47 | 9 |
| Spironolactone Meta | 341.1 | 107.1 | 4 | 165 | 47 | 9 |
| Telmisartan Meta | 691.4 | 515.4 | 4 | 165 | 47 | 9 |
| Aspirin-1 | 163.2 | 64.9 | 4 | 42 | 39 | 10 |
| Aspirin-2 | 163.2 | 120.8 | 4 | 43 | 17 | 7 |
| Bumetanide | 365.2 | 240.1 | 4 | 128 | 24 | 18 |
| Furosemide | 331 | 80.9 | 4 | 131 | 26 | 8 |
| Hydrochlorothiazide | 297.3 | 98.9 | 4 | 168 | 40 | 12 |

(8) Detection of aldosterone: for sample pretreatment of aldosterone detection in plasma, supportive liquid-liquid extraction (SLE) was used for extraction, and liquid chromatography-tandem mass spectrometry was used for detection; specific steps were as follows: 300-450 μL plasma was transferred and added to 50 μL aldosterone internal standard solution, samples were loaded on an SLE plate, standing for 10 min, and eluted by a 1.5 mL (2*750 ul) mixed solvent of ethyl acetate and n-hexane (1:1). After eluent was dried, the resident was redissolved and detected by liquid chromatography-tandem mass spectrometer; an ESI+detection mode was taken; quantitative MRM ion pair of Aldosterone was 361.3/315.1; MRM ion pair of an Aldosterone internal standard Aldosterone-d8 was 369.4/323.1; and the representative detection chromatogram was shown in FIG. 2. A great number of studies have proved that the 43 medicaments influencing ARR cannot be synchronously screened during the pretreatment and detection of aldosterone; and the reason is probably that the pretreatment process is not suitable for the extraction of all 43 medicaments influencing ARR; and the experimental data is omitted.

(9) Test report of the medicament screening; in the plasma PA screening report (as shown in FIG. 3), the screening results of the medicament factors leading to false positive or false negative ARR were collected and in clinical combination with ARR values and medicament screening results to effectively eliminate partial false positive or false negative results caused by medicament interference.

### EXAMPLE 2: VERIFICATION OF FALSE NEGATIVE CLINICAL SAMPLES

In this example, clinical samples were detected for PA Screening; and the detection method used in Example 1 was taken for ARR detection and qualitative judgment; and the detection results of the clinical samples were shown in Table 5.

TABLE 5
Detection results of clinical samples

| | | Unit of | |
|---|---|---|---|
| Item | Result | measurement | Reference interval |
| Aldosterone | 222.46 | pg/ml | ≥18 years old: |
| [ALD] | | | Upright position: |
| | | | 8:00-10:00 A.M. ≤280, |
| | | | 4:00-6:00 P.M. ≤210; |
| | | | Supine position: |
| | | | 8:00-10:00 |
| | | | A.M. 30-160 |
| Aldosterone/renin | 28.78 | | |
| activity ratio | | | |
| [ARR] | | | |
| Angiotensin I | 2.52 | ng/ml | |
| (after incubation) | | | |
| Angiotensin I | 0.21 | ng/ml | |
| (before incubation) | | | |
| Plasma Renin | 0.77 | ng/ml/h | 0.25-5.82 |
| Activity [PRA] | | | |

Based on the above detection results of the sample, ARR was less than the cut-off value (30) of disease diagnosis; therefore, the patient in the sample was judged as negative. But, in combination with the screening results of antihypertensive medicaments of the patient in Table 4, ARB and CCB medicaments were detected as positive, that is, both might lead to a false negative result. Through comprehensive assessment, the ARR result of the patient sample was lower than the cut-off value, the patient also might be judged as positive, and recommended to receive a further experiment for confirming diagnosis.

TABLE 6
Patient's medicament screening result

| | | | | Influence | |
|---|---|---|---|---|---|
| | Screening | Name of | Influence on | on Renin | Influence |
| Medicament factors | results | medicament | Aldosterone | Activity | onARR |
| β-receptor blocker | − | No | ↓ | ↓↓ | ↑(False positive) |
| Centralα2receptor agonist | − | No | ↓ | ↓↓ | ↑(False positive) |
| NSAID | − | No | ↓ | ↓↓ | ↑(False positive) |
| Potassium-removing diuretics | − | No | →↑ | ↑↑ | ↓(False negative) |
| Potassium-retaining diuretics | − | No | ↑ | ↑↑ | ↓(False negative) |
| ACEI | − | No | ↓ | ↑↑ | ↓(False negative) |
| ARBs | + | Valsartan | ↓ | ↑↑ | ↓(False negative) |
| Dihydropyridine CCBs | + | Amlodipine | →↓ | ↑ | ↓(False negative) |

The patient was definitely diagnosed with primary aldosteronism via a Captopril experiment for a confirmation diagnosis.

### EXAMPLE 3: DETECTION AND CONFIRMATION OF FALSE NEGATIVE CLINICAL SAMPLES

In this example, clinical samples were detected for PA screening; and the detection method used in Example 1 was taken for ARR detection and qualitative judgment; and the detection results of the clinical samples were shown in Table 7.

TABLE 7
Detection results of clinical samples

| | | Unit of | Reference |
|---|---|---|---|
| Item | Result | measurement | interval |
| Aldosterone | 134.32 | pg/ml | ≥18 years old: |
| [ALD] | | | Upright position: |
| | | | 8:00-10:00 A.M. ≤280, |
| | | | 4:00-6:00 P.M. ≤210; |
| | | | Supine position: |
| | | | 8:00-10:00 |
| | | | A.M. 30-160 |
| Aldosterone/renin | 33.58 | | |
| activity ratio | | | |
| [ARR] | | | |
| Angiotensin I (after | 1.20 | ng/ml | |
| incubation) | | | |
| Angiotensin I (before | <0.2 | ng/ml | |
| incubation) | | | |
| Plasma Renin | 0.40 | ng/ml/h | 0.25-5.82 |
| Activity [PRA] | | | |

Based on the above detection results of the sample, ARR was higher than the cut-off value (30) of disease diagnosis; therefore, the sample was judged as positive. But, in combination with the screening results of antihypertensive medicaments in Table 8, the β-receptor blocker and central α2 receptor agonist were detected as positive, while both might lead to a false negative result. Through comprehensive assessment, the ARR result of the patient sample was higher than the cut-off value, the patient also might be judged as negative, and recommended to receive a further experiment for a definite diagnosis.

TABLE 8
Patient's medicament screening result

| | | | | Influence | |
|---|---|---|---|---|---|
| | Screening | Name of | Influence on | on renin | Influence |
| Medicament factors | results | medicament | aldosterone | activity | on ARR |
| β-receptor blocker | + | Metoprolol | ↓ | ↓↓ | ↑(False positive) |
| Centralα2receptor agonist | + | Methyldopa | ↓ | ↓↓ | ↑(False positive) |
| NSAID | − | − | ↓ | ↓↓ | ↑(False positive) |
| Potassium-removing diuretics | − | − | →↓ | ↑↑ | ↓(False negative) |
| Potassium-retaining diuretics | − | − | ↑ | ↑↑ | ↓(False negative) |
| ACEI | − | − | ↓ | ↑↑ | ↓(False negative) |
| ARBs | − | − | ↓ | ↑↑ | ↓(False negative) |
| Dihydropyridine CCBs | − | − | →↓ | ↑ | ↓(False negative) |

The patient was diagnosed as negative after Captopril confirmation test.

### EXAMPLE 4: DETECTION AND CONFIRMATION OF POSITIVE CLINICAL SAMPLES

In this example, clinical samples were detected for PA screening; and the detection method used in Example 1 was taken for ARR detection and qualitative judgment; and the detecting results of the clinical samples were shown in Table 9.

TABLE 9
Detection results of the clinical samples

| | | Unit of | Reference |
|---|---|---|---|
| Item | Result | measurement | interval |
| Aldosterone | 201.53 | pg/ml | ≥18 years old: |
| [ALD] | | | Upright position: |
| | | | 8:00-10:00 |
| | | | A.M. ≤280, |
| | | | 4:00-6:00 |
| | | | P.M. ≤210; |
| | | | Supine position: |
| | | | 8:00-10:00 |
| | | | A.M.30-160 |
| Aldosterone/renin | 49.15 | | |
| activity ratio | | | |
| [ARR] | | | |
| Angiotensin I (after | 1.58 | ng/ml | |
| incubation) | | | |
| Angiotensin I (before | 0.35 | ng/ml | |
| incubation) | | | |
| Renin Renin | 0.41 | ng/ml/h | 0.25-5.82 |
| Activity[PRA] | | | |

Based on the above detection results of the sample, ARR was higher than the cut-off value (30); therefore, the patient was judged as positive. Meanwhile, in combination with the screening results of antihypertensive medicaments of the patient in Table 10, ARB and potassium-removing diuretics were detected as positive, that is, both might lead to a false negative result of the detected ARR. Through comprehensive assessment, positive ARB and potassium-removing diuretics might lead to false negative, while the ARR result was still higher than the cut-off value, the patient might be judged as a positive sample, therefore the patient was recommended to receive a further confirmation experiment.

TABLE 10
Patient's medicament screening result

| | Screening | Name of | Influence on | Influence on | Influence |
|---|---|---|---|---|---|
| Medicament factors | results | medicament | aldosterone | renin activity | on ARR |
| β-receptor blocker | − | − | ↓ | ↓↓ | ↑(False positive) |
| Centralα2receptor agonist | − | − | ↓ | ↓↓ | ↑(False positive) |
| NSAID | − | − | ↓ | ↓↓ | ↑(False positive) |
| Potassium-removing diuretics | + | Hydrochlorothiazide | →↑ | ↑↑ | ↓(False negative) |
| Potassium-retaining diuretics | − | − | ↑ | ↑↑ | ↓(False negative) |
| ACEI | − | − | ↓ | ↑↑ | ↓(False negative) |
| ARBs | + | Candesartan | ↓ | ↑↑ | ↓(False negative) |
| Dihydropyridine CCBs | − | − | →↓ | ↑ | ↓(False negative) |

Even though the present invention is disclosed above, the present invention is not limited thereto. A person skilled in the art can make various alterations and modifications within the spirit and scope of the present invention. Therefore, the protection scope of the present invention should be subjected to the scope defined by the claims.

1. A method for synchronous qualitative detection of medicaments influencing ARR in the detection process of renin activity by liquid chromatography-tandem mass spectrometry, comprising: detecting a production rate of angiotensin I and hypertension therapeutics influencing ARR in a plasma sample; combining a concentration of aldosterone to obtain ARR values, thus analyzing and judging ARR as one of a negative, positive, false negative or false positive results.

2. The detection method of claim 1, wherein the hypertension therapeutics influencing ARR, including 9 β-receptor blockers, 5 potassium-removing diuretics, 3 potassium-retaining diuretics, 10 angiotensin converting enzyme inhibitors, 6 angiotensin receptor antagonists, 8 calcium channel blockers, 1 central α2 receptor agonist and 1 non-steroid anti-inflammatory drug.

3. The detection method of claim 2, wherein the ARR has a computing formula as follows:
ARR=concentration of aldosterone/production rate of angiotensin I.

4. The detection method of claim 3, wherein the hypertension therapeutics influencing ARR are:
the 9 α-receptor blockers comprising Arotinolol, Atenolol, Bisoprolol, Esmolol, Labetalol, Metoprolol, Nebivolol, Propranolol, and Sotalol;
the 5 potassium-removing diuretics comprising Bumetanide, Furosemide, Hydrochlorothiazide, Indapamide, and Torasemidel;
the 3 potassium-retaining diuretics comprising Amiloride, Eplerenone, and Spironolactone;
the 10 angiotensin converting enzyme inhibitors comprising Benazepril, Captopril, Enalapril, Fosinopril, Imidapril, Lisinopril, Perindopril, Quinapril, Ramipril, and Trandolapril;
the 6 angiotensin receptor antagonists comprising Candesartan, Irbesartan, Losartan, Olmesartan, Telmisartan, and Valsartan;
the 8 calcium ion antagonists comprising Amlodipine, Benidipine, Felodipine, Flunarizine, Lercanidipine, Nicardipine, Nifedipine, and Nimodipine;
the 1 central α2 receptor agonist comprising Methyldopa; and
the 1 non-steroid anti-inflammatory drug comprising aspirin.

5. The detection method of claim 4, wherein the β-receptor blockers, the central α2 receptor agonist, and the non-steroid anti-inflammatory drug can increase ARR to show a false positive result; the potassium-removing diuretics, the potassium-retaining diuretics, the angiotensin converting enzyme inhibitors, the angiotensin receptor antagonists, and the calcium channel blockers can decrease ARR to show a false negative result;
the procedure for analysis of ARR values are as follows: analyzing and judging ARR as one of a negative, positive, false negative or false positive results by a clinical medication AI guidance system, wherein the clinical medication AI guidance system comprises a patient information module, a database module and a decision tree system.

6. The detection method of claim 5, wherein the information module is used to record a patient's basic information; the database module is used to store detection results of ARR and detection results of hypertension therapeutics; and the decision tree system automatically analyzes and judges an ARR index as one of a negative, positive, false negative or false positive result according to the detection results.

7. The detection method of any one of claim 6, wherein the analysis and judgment method of the decision tree system is as follows: analyzing and judging the ARR indices as one of a negative, positive, false negative or false positive results according to the qualitative detecting results of the medicaments influencing ARR, wherein when the ARR detection result is less than 30, and meanwhile if the patient is detected to contain a medicament capable of decreasing ARR, the ARR indices may be judged as false negative, and an experiment for confirmation is further needed; when the ARR detection result is less than 30, and meanwhile medicament capable of increasing ARR positive, the ARR indices may be judged as false positive, and an experiment for conformation is necessary; and when the ARR detection result is higher than 30, and meanwhile if the patient is detected to contain a medicament capable of decreasing ARR, the ARR indices may be judged as positive.

8. The detecting method of claim 7, wherein the production rate of the angiotensin I is obtained by detecting concentrations of angiotensin I in the sample before and after incubation respectively and calculated according to the following formula: production rate of angiotensin I=(concentration of angiotensin I after incubation−concentration of angiotensin I before incubation)/incubation time.

9. The detection method of claim 8, wherein the detecting procedures are as follows:
(1) sample pretreatment: 1) taking two parts of 100 μL parallel samples, adding a generation buffer solution to the samples respectively and mixing evenly, subjecting one part to incubation, and subjecting another part to next step directly without incubation; 2) adding a terminating solution to samples where the generation buffer solution is added, and mixing evenly; 3) adding a protein precipitant with internal standard to the samples where the terminating solution is added in the step 2), and vortex mixing evenly; and 4) mixing the samples where the protein precipitant is added in step 3) and then performing centrifugation, and taking supernatant for detection on an instrument;
(2) performing the detection of angiotensin I and screening of 43 hypertension therapeutics influencing ARR with sample before incubation obtained in the above steps with a high performance liquid chromatography-tandem mass spectrometry system;
(3) respecting to the hit for medicament screening result, a double-check is necessary with simultaneous reference to the retention time of the standard substance of each hypertension therapeutic in a “mixed medicament-screening working solution”, thus ensuring the accuracy of the hit of the medicament screening from two dimensions of mass spectrum parameters and retention time.

10. The detection method of claim 9, wherein the protein precipitant with internal standard is prepared by: methanol, a zinc sulfate aqueous solution and an angiotensin I isotope internal standard.

11. The detection method of claim 9, wherein the generation buffer solution is: a PMSF-containing Tris and EDTA buffer solution; and the terminating solution is: formic acid or acetic acid.

12. The detection method of claim 9, wherein the mixed medicament-screening working solution is a mixed medicament-screening working solution prepared by standard substances of the 43 hypertension therapeutics influencing ARR.

13. The detection method of claim 9, wherein the incubation refers that a sample is added to a generation buffer solution and mixed evenly, then put to a 37° C. water bath for 3 h;
and a non-incubated sample is used for detecting Angiotensin I before incubation and screening the 43 hypertension therapeutics influencing ARR.

14. The detection method of claim 9, wherein mobile phase A of the liquid chromatography is 0.1% formic acid aqueous solution, mobile phase B is methanol solution with 0.1% formic acid; and the gradient is shown in the Table below:

| Time | Flow rate | Mobile phase | Mobile phase |
|---|---|---|---|
| (min) | (mL/min) | A (%) | B (%) |
| 0 | 0.6 | 90 | 10 |
| 1 | 0.6 | 90 | 10 |
| 2 | 0.6 | 5 | 95 |
| 3.2 | 0.6 | 5 | 95 |
| 3.25 | 0.6 | 90 | 10 |
| 3.8 | 0.6 | 90 | 10 |

15. The detection method of claim 9, wherein the retention time of the standard substance of each hypertension therapeutic in the “mixed medicament-screening working solution” is shown in the table below:

| | | Limit of | |
|---|---|---|---|
| | | detection | |
| | | (LOD) of | |
| | English name | medicament | Retention |
| Type of medicament | of medicament | (ng/mL) | time (min) |
| Potassium-retaining | Amiloride | 25 | 1.06 |
| diuretics | | | |
| CCBs | Amlodipine | 25 | 2.19 |
| β-receptor blocker | Arotinolol | 5 | 1.97 |
| Non-steroid | aspirin | 250 | 2.33 |
| anti-inflammatory drug | | | |
| β-receptor blocker | Atenolol | 25 | 0.65 |
| ACEI | Benazepril | 5 | 2.19 |
| CCBs | Benidipine | 5 | 2.16 |
| β-receptor blocker | Bisoprolol | 5 | 2.05 |
| Potassium-removing | Bumetanide | 25 | 2.29 |
| diuretics | | | |
| ARBs | Candesartan | 25 | 2.32 |
| ACEI | Captopril | 100 | 1.95 |
| ACEI | Enalapril | 5 | 2.1 |
| Potassium-retaining | Eplerenone | 25 | 2.16 |
| diuretics | | | |
| β-receptor blocker | Esmolol | 5 | 1.98 |
| CCBs | Felodipine | 250 | 2.39 |
| CCBs | Flunarizine | 5 | 2.25 |
| ACEI | Fosinopril | 100 | 2.46 |
| Potassium-removing | Furosemide | 250 | 2.61 |
| diuretics | | | |
| Potassium-removing | Hydrochlorothia | 250 | 1.97 |
| diuretics | zide | | |
| ACEI | Imidapril | 5 | 2.13 |
| Potassium-removing | Indapamide | 25 | 2.17 |
| diuretics | | | |
| ARBs | Irbesartan | 5 | 2.27 |
| β-receptor blocker | Labetolol | 25 | 2.04 |
| CCBs | Lercanidipine | 5 | 2.26 |
| ACEI | Lisinopril | 25 | 1.77 |
| ARBs | Losartan | 5 | 2.26 |
| Centralα2 receptor | Methyldopa | 250 | 0.39 |
| agonist | | | |
| β-receptor blocker | Metoprolol | 25 | 1.94 |
| β-receptor blocker | Nebivolol | 25 | 2.2 |
| CCBs | Nicardipine | 5 | 2.15 |
| CCBs | Nifedipine | 250 | 2.27 |
| CCBs | Nimodipine | 25 | 2.34 |
| ARBs | Olmesartan | 25 | 2.12 |
| ACEI | Perindopril | 5 | 2.14 |
| β-receptor blocker | Propranolol | 25 | 2.08 |
| ACEI | Quinapril | 5 | 2.22 |
| ACEI | Ramipril | 5 | 2.2 |
| β-receptor blocker | Sotalol | 25 | 0.53 |
| Potassium-retaining | Spironolactone | 250 | 2.29 |
| diuretics | | | |
| ARBs | Telmisartan | 25 | 2.24 |
| Potassium-removing | Torasemide | 5 | 2.13 |
| diuretics | | | |
| ACEI | Trandolapril | 5 | 2.25 |
| ARBs | Valsartan | 100 | 2.34 |

16. The detection method of claim 9, wherein during the mass spectrometric detection, a triple quadrupole mass spectrometer with a model of SCIEX 4500MD is used; and the mass spectrometric detection is performed by adopting a positive ion mode of an electrospray ionization ion source (ESI+) and a multiple-reaction monitoring MRM mode; and the corresponding mass spectrometric detection method is configured as shown in the following table:

| Mass spectrometry | | |
|---|---|---|
| conditions | Value | |
| Curtain gas CUR | 30 | psi |
| Atomization gas GS1 | 50 | psi |
| Auxiliary heating gas GS2 | 50 | psi |
| Heating temperature of ion | 500° | C. |
| sources | | |
| Collision gas CAD | 10 | psi |
| Spray voltage | 5500 | V |

17. The detection method of claim 16, wherein during the mass spectrometric detection, mass spectrum parameters of each substance to be detected are shown in the table below:

| | | | Dwell | | | |
|---|---|---|---|---|---|---|
| Compound | Q1 | Q3 | Time | DP | CE | CXP |
| ANG I-1 | 433.1 | 647.6 | 30 | 74 | 24 | 12 |
| ANG I-2 | 433.1 | 619.6 | 5 | 87 | 26 | 12 |
| ANG I-IS | 437.3 | 660.4 | 30 | 70 | 22 | 12 |
| Metoprolol | 268.1 | 133 | 4 | 90 | 37 | 9 |
| Esmolol | 296.1 | 145.1 | 4 | 78 | 38 | 9 |
| Labetolol | 329.2 | 162 | 4 | 78 | 35 | 9 |
| Bisoprolol | 326.5 | 116.2 | 4 | 96 | 25 | 9 |
| Flunarizine | 405.2 | 203 | 4 | 79 | 21 | 9 |
| Ramipril | 417.3 | 234.1 | 4 | 78 | 29 | 9 |
| Lercanidipine | 612.2 | 280.2 | 4 | 94 | 31 | 9 |
| Benazepril | 425.2 | 351.1 | 4 | 105 | 30 | 9 |
| Quinapril | 439.1 | 234 | 4 | 90 | 27 | 9 |
| Losartan | 423.2 | 207 | 4 | 69 | 33 | 9 |
| Trandolapril | 431.2 | 234 | 4 | 90 | 31 | 9 |
| Telmisartan | 515.4 | 276 | 4 | 153 | 59 | 9 |
| Nifedipine-1 | 347.1 | 315.1 | 4 | 79 | 13 | 9 |
| Nifedipine-2 | 347.3 | 195.1 | 4 | 65 | 58 | 6 |
| Nicardipine | 480.2 | 315.1 | 4 | 78 | 34 | 9 |
| Benidipine | 506.2 | 174 | 4 | 78 | 37 | 9 |
| Torasemide | 349.1 | 264.1 | 4 | 63 | 22 | 9 |
| Nebivolol | 406.1 | 151 | 4 | 105 | 41 | 9 |
| Indapamide | 366 | 132 | 4 | 78 | 20 | 9 |
| Imidapril | 406.2 | 234 | 4 | 75 | 27 | 9 |
| Eplerenone | 415.2 | 162.9 | 4 | 107 | 28 | 9 |
| Olmesartan | 447.1 | 207 | 4 | 83 | 37 | 9 |
| Irbesartan | 429.4 | 206.8 | 4 | 108 | 38 | 9 |
| Nimodipine | 419.2 | 343.1 | 4 | 82 | 13 | 9 |
| Valsartan-1 | 436.3 | 206.9 | 4 | 90 | 38 | 9 |
| Valsartan-2 | 436.4 | 207.2 | 4 | 90 | 38 | 4 |
| Felodipine | 384 | 338 | 4 | 76 | 13 | 9 |
| Spironolactone | 417.2 | 341.2 | 4 | 90 | 12 | 9 |
| Candesartan | 441.1 | 263 | 4 | 97 | 18 | 9 |
| Atenolol | 267.2 | 145 | 4 | 78 | 35 | 9 |
| Methyldopa | 212 | 166 | 4 | 75 | 21 | 9 |
| Amiloride | 230.1 | 170.9 | 4 | 69 | 24 | 9 |
| Sotalol | 273.1 | 133 | 4 | 70 | 36 | 9 |
| Arotinolol | 372 | 316.1 | 4 | 78 | 22 | 9 |
| Lisinopril | 406.2 | 84 | 4 | 78 | 52 | 9 |
| Enalapril | 377.2 | 234.1 | 4 | 70 | 26 | 9 |
| Perindopril | 369.1 | 172.1 | 4 | 90 | 31 | 9 |
| Propranolol | 260 | 116 | 4 | 90 | 24 | 9 |
| Amlodipine-1 | 409 | 237.9 | 4 | 69 | 12 | 9 |
| Amlodipine-2 | 409.2 | 206 | 4 | 55 | 37 | 10 |
| Fosinopril | 564.4 | 492.2 | 4 | 84 | 11 | 9 |
| Captopril-1 | 218.1 | 116.1 | 4 | 73 | 20 | 9 |
| Captopril-2 | 218.2 | 70 | 4 | 70 | 25 | 6 |
| Nifedipine Meta-1 | 331.2 | 270.2 | 4 | 165 | 47 | 9 |
| Felodipine Meta | 384 | 320 | 4 | 165 | 47 | 9 |
| Spironolactone Meta | 341.1 | 107.1 | 4 | 165 | 47 | 9 |
| Telmisartan Meta | 691.4 | 515.4 | 4 | 165 | 47 | 9 |
| Aspirin-1 | 163.2 | 64.9 | 4 | 42 | 39 | 10 |
| Aspirin-2 | 163.2 | 120.8 | 4 | 43 | 17 | 7 |
| Bumetanide | 365.2 | 240.1 | 4 | 128 | 24 | 18 |
| Furosemide | 331 | 80.9 | 4 | 131 | 26 | 8 |
| Hydrochlorothiazide | 297.3 | 98.9 | 4 | 168 | 40 | 12 |

18. The detection method of claim 1, wherein influences of the hypertension medicaments on ARR can be analyzed and predicted by AI software.

19. A kit for synchronous qualitative detection of antihypertensive medicaments influencing ARR in the detection process of renin activity by liquid chromatography-tandem mass spectrometry, the kit comprising a protein precipitant containing an internal standard, a generation buffer solution, a terminating solution, a mixed medicament-screening working solution and liquid mobile phases, wherein the protein precipitant with internal standard is prepared by methanol, a zinc sulfate aqueous solution and an angiotensin I isotope-labeled internal standard; the generation buffer solution is: a PMSF-containing Tris and EDTA buffer solution; and the terminating solution is formic acid or acetic acid; the mixed medicament-screening working solution is a mixed medicament-screening working solution prepared by standard substances of 43 hypertension therapeutics influencing ARR at certain concentration; mobile phase A of the liquid chromatography is a 0.1% formic acid aqueous solution, and mobile phase B is a methanol solution with of 0.1% formic acid.
